(19) United States
(12) Patent Application Publication
(10) Pub. No.: US 2015/0238446 A1
(43) Pub. Date: Aug. 27, 2015

(54) NEW TREATMENT OF FISH WITH A NANOSUS PENS ION OF LUFENURON OR HEXAFLUMURON

(71) Applicant: Novartis AG, Basel (CH)

(72) Inventor: Caroline Kablitz, Basel (CH)

(21) Appl. No.: 14/416,093

(22) PCT Filed: Jul. 22, 2013
(86) PCT No.: PCT/EP2013/065422

§ 371 Date: Jan. 21, 2015

(30) Foreign Application Priority Data
Jul. 27, 2012 (EP) 12178186.8

(51) Int. Cl. A61K 31/17; A61K 9/00; A61K 9/10

(52) U.S. Cl. CPC A61K 31/17; A61K 9/10; A61K 9/0019

(57) ABSTRACT

The present invention concerns a stable aqueous nanosuspension dosage formulation for injection to fish, comprising (i) an active ingredient which is lufenuron or hexaflumuron, each in form of particles having an effective average particle size of ≦1000 nm; (ii) one or more surface stabilizers selected from the group consisting of a surfactant and a polymer or a mixture thereof; and (iii) water. The nanosuspension formulations of the present invention are especially suited for the treatment of salmon and provide prolonged effective protection against sea lice at sea.

The present invention relates to a stable nanosuspension dosage composition comprising a chitin synthesis inhibitor, in particular lufenuron or hexaflumuron, and its use as injectable in the control of sea lice, for example Lepeophtheirus salmonis, Caligus elongatus and Caligus rogercresseyi, infestations in fish farming.

The basis of sea lice control in commercial salmonid farming is largely still a treatment with chemicals such as organophosphates, synthetic pyrethroids, chitin synthesis inhibitors, hydrogen peroxide or macrocyclic lactones such as emamectin benzoate. Developing resistance by sea lice against said commercial products presents a big threat to the fish industry; on the one hand higher doses of the compounds might be employed which accelerates the issue of resistance development and moreover has the potential to create environmental toxicology issues. On the other hand there is a desperate search for new chemicals and treatment schedules.

WO99/063824 discloses the use of hexaflumuron and lufenuron, both members of the chemical class of benzoylureas, in the control of sea lice infestations on fish. Hexaflumurone is applied to the fish orally, via bath treatment or via injection, lufenuron is proposed exclusively for injection. Oily injectable formulations of the two active ingredients, in particular those comprising, in addition, a vaccine against furunculosis, show activity against sea lice for 128 days or in case of hexaflumuron even longer. However, stability testing of oily lufenuron or hexaflumuron formulations has revealed that the active ingredients are chemically instable in organic solvents/media. The resulting low shelf life make them unsuited for commercial use. WO99/063824 further discloses a hexaflumuron containing aqueous suspension, which shows, however, a poor performance when tested in vivo against sea lice, thus indicating a low bioavailability. In addition, as both hexaflumuron and lufenuron have a strong tendency to aggregate and form sediments in an aqueous environment, the handling of water-based injectables is difficult and cumbersome. So it is not surprising that no commercial lufenuron or hexaflumuron sea lice product has made it to the market until now.

In view of the high potential of lufenuron and hexaflumuron, the provision of an improved injectable formulation, which is chemically stable over time, does not aggregate or form sediments, and provides high long-term efficacy against sea lice, would be highly desirable.

Surprisingly, it has now been found that a very long-lasting and convenient control of sea lice infesting fish, and in particular salmon, may be obtained by applying an improved lufenuron- or hexaflumuron-containing injectable formulation to the fish which is in the form of an aqueous nanosuspension.

Therefore, the present invention, according to one aspect, relates to a stable aqueous nanosuspension dosage formulation for injection to fish, comprising

(i) an active ingredient which is lufenuron or hexaflumuron, each in form of particles having an effective average particle size of 51000 nm;

(ii) one or more surface stabilizers selected from the group consisting of a surfactant and a polymer or a mixture thereof; and

(iii) water.

The present invention further concerns a method for eliminating, reducing or preventing sea lice in a fish population, which comprises injecting the fish with an above-mentioned aqueous nanosuspension dosage formulation comprising lufenuron or hexaflumuron.

When applying a treatment according to the present invention, therapeutically effective lufenuron concentrations in the blood, fillet and skin of the fish may be obtained for at least 5 months (150 days), thus indicating an effective protection of the fish against sea lice for a prolonged period of time.

### DETAILED DESCRIPTION

Lufenuron, N-{[2,5-Dichlor-4-(1,1,2,3,3,3-hexafluorpropoxyl)phenyl]carbamoyl}-2,6-difluorbenzamid, has the chemical formula

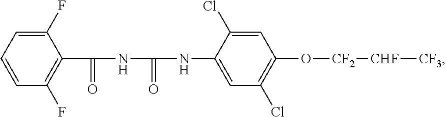

and may be applied in free form or in form of a veterinary acceptable salt. Due to an asymmetrical C-atom being present in the molecule, two enantiomers are in existence. Within the present invention the use of lufenuron in form of the racemic mixture of the two enantiomers is preferred. In addition, racemic lufenuron may exist in various polymorphic forms, for example as polymorph A, B, C or D. Within the present invention, the use of the thermodynamically most stable polymorph A is preferred.

Hexaflumuron, 1-[3,5-dichloro-4-(1,1,2,2-tetrafluoroethoxyl)phenyl]-3-(2,6-difluorobenzoyl)urea, has the formula

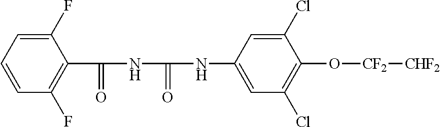

and may be applied in free form or in form of a veterinary acceptable salt.

The water-solubility of both active ingredients, ≦0.027 mg/l for hexaflumuron and ≦0.06 mg/l for lufenuron is very low. Within the present invention aqueous formulations are provided, wherein the active ingredient particles have an average particle size of ≦1000 nm, preferably ≦900 nm, more preferably ≦750 nm and in particular ≦600 nm, especially from 10 to 600 nm. A suitable average active ingredient particle range is preferably 10 to 900 nm, more preferably 50 to 750 nm and in particular from 90 to 600 nm.

The concentration of the active ingredient in the nanosuspension formulation may vary within a wide range, but is suitably from 1 to 20% (w/v), preferably from 2 to 10% (w/v), and in particular from 3 to 8% (w/v), based on the entire aqueous dosage formulation.

One embodiment of the present invention comprises a stable aqueous nanosuspension dosage formulation comprising lufenuron, wherein the above and below given meanings and preferences apply.

A further embodiment of the present invention comprises a stable aqueous nanosuspension dosage formulation comprising hexaflumuron, wherein the above and below given meanings and preferences apply.

Suitable surfactants for surface stabilization of the active ingredient are for example non-ionic, anionic or zwitterionic surfactants. Suitable non-ionic, anionic or zwitterionic surfactants are known in high number and are commercially available.

Representative examples include but are not limited to:

Non-ionic surfactants: Fatty alcohols, for example cetyl alcohol, stearyl alcohol or cetostearyl alcohol; sorbitan fatty acid esters, for example sorbitan monopalmitate, sorbitan monostearate, sorbitan sesquioleate or sorbitan trioleate (Span™ 40, 60, 83 or 85); polyoxyethylene sorbitan fatty acid esters (Tween® 20-Tween® 80), in particular polyoxyethylene(20)-sorbitan monolaurate, polyoxyethylene(20)-sorbitan monopalmitate, polyoxyethylene(20)-sorbitan monostearate or polyoxyethylene(20)-sorbitan monooleate; polyethoxylated castor oils (Cremophor® EL/ELP), in particular the reaction product of castor oil and ethylene oxide in a molar ratio of about 1:35; polyoxyethylene esters of fatty acids, for example polyoxyethylene stearates or polyoxyethylene 12-hydroxy-stearates (Macrogol stearate 2000, Solutol® HS); glycerol monostearate; poloxamers which are block copolymers of polyethylene oxide and polypropylene oxide (poloxamer 188, poloxamer 407, Pluronic® F68, F127), in particular poloxamers having an average molecular weight M_{w} of from about 1500 to about 4000 and having an polyoxyethylene/polyoxypropylene ratio from about 70/30 to about 80/20.

Ionic surfactants: an organic sulfonic acid comprising, for example, from 8 to 24 C-atoms or a salt thereof, for example sodium cetylstearyl sulphate, sodium lauryl sulphate, sodium dodecyl sulphate, sodium dioctylsulfosuccinate; a fatty acid comprising, for example, from 4 to 28 C-atoms or a salt thereof, for example oleic acid, sodium oleate, sodium deoxycholate, stearic acid, calcium stearate.

Zwitter-ionic surfactants: lecithin, hydroxylated lecithin, phosphatidyl cholines (Phospholipon® 90).

A preferred surfactant for surface stabilization is sodium dodecyl sulphate, sodium dioctylsulfosuccinate, a polyethoxylated castor oil, a polyoxyethylene sorbitan fatty acid ester or a poloxamer

An especially preferred surfactant for surface stabilization of the active ingredients is sodium dodecyl sulphate, sodium dioctylsulfosuccinate, the reaction product of castor oil and ethylene oxide in a molar ratio of about 1:35, polyoxyethylene(20)-sorbitan monolaurate, polyoxyethylene(20)-sorbitan monopalmitate, or a poloxamer having an average molecular weight M_{w} of from about 1500 to about 4000 and having an polyoxyethylene/polyoxypropylene ratio from about 70/30 to about 80/20.

Representative examples of surface stabilizing polymers include but are not limited to: Polyvinylpyrrolidones (PVP K12, PVP K17, PVP K30), in particular polyvinylpyrrolidones having an average molecular weight M_{w} of from about 2500 to 50000; polyvinyl alcohols, for example those having an average molecular weight M_{w} of from 7500 to 40000; polylactic acids; polylactic-co-glycolic acids; polyethylene glycols, in particular polyethylene glycols 300-1500; natural and modified celluloses, for example microcrystalline cellulose, sodium carboxymethylcellulose, methylcellulose, hydroxypropylmethylcellulose; starches, for example hydroxyethyl starch polysaccharides, for example dextrans, cyclodextrins, chitosanes; polydimethylsiloxanes; silicon dioxides; tragacanth; albumin; hydrolyzed gelatin, cholesterol.

A preferred polymer for surface stabilization of the active ingredients is a polyvinyl alcohol (PVA) or a polyvinyl pyrrolidone (PVP). An especially preferred polymer for surface stabilization of the active ingredients is a PVA having an average molecular weight M_{w} of from 7500 to 40000 or a PVP having an average molecular weight M_{w} of from about 2500 to 50000.

According to one embodiment of the invention the active ingredient particles are stabilized with one surface stabilizer selected from a surfactant and a polymer, wherein the above given meanings and preferences apply.

According to a further embodiment of the invention the active ingredient particles are stabilized with two or more, in particular two, surface stabilizers selected from a surfactant and a polymer, wherein the above given meanings and preferences apply.

According to a preferred embodiment of the invention the active ingredient particles are stabilized with a combination of one or more surfactants and one or more polymers, in particular with one surfactant and one polymer each, wherein for the surfactants and polymer each the above given meanings and preferences apply.

According to a further preferred embodiment, the surface stabilizer comprises one or more polymers and/or polymers selected from the group consisting of sodium dodecyl sulphate, sodium dioctylsulfosuccinate, a reaction product of castor oil and ethylene oxide in a molar ratio of about 1:35, polyoxyethylene(20)-sorbitan monolaurate, polyoxyethylene(20)-sorbitan monopalmitate, a poloxamer having an average molecular weight M_{w} of from 1500 to 4000 and having an polyoxyethylene/polyoxypropylene ratio from 70/30 to 80/20, a polyvinyl alcohol (PVA) having an average molecular weight M_{w} of from 7500 to 40000 and a polyvinyl pyrrolidone (PVP) having an average molecular weight M_{w} of from about 2500 to 50000.

The total amount of surface stabilizers in the nanosuspension formulation may vary within a wide range, but is suitably from 0.5 to 20% (w/v), preferably from 0.5 to 10% (w/v), more preferably from 1.0 to 8% (w/v), and in particular from 1 to 4% (w/v), in each case based on the entire formulation.

The nanosuspension formulations of the present invention may contain additional excipients, for example:
a crystal growth inhibitor, for example, selected from the group consisting of a mono- or disaccharide, a sugar alcohol, a glycol, glycerol, an at least partly water-soluble salt, and mixtures thereof. Examples are dextrose, sucrose, fructose, mannose, lactose, trehalose; mannitol, sorbitol, xylitol; ethylene glycol, propylene glycol; glycerol; sodium chloride, potassium chloride, magnesium chloride. Preferred crystal inhibitors are mannitol, sorbitol, dextrose, lactose, sucrose, trehalose, propylene glycol, glycerol and sodium chloride. The amount of crystal growth inhibitor in the nanosuspensions of the present invention may be, for example, from 0 to 20% (w/v); if present, an amount of from 0.1% to 20% (w/v), preferably from 0.25 to 15% (w/v), and in particular from 0.5 to 10% w/v) of crystal growth inhibitor or mixture of different crystal growth inhibitors, based on the entire formulation, has proven as valuable.
a preservative. Suitable preservatives are known per se and encompass, for example, chlorobutanol, benzylalcohol, m-cresol, a paraben or mixture of different parabens, such as a C₁-C₅-alkylparaben, in particular methylparaben or propylparaben, phenol, sorbic acid, phenoxyethanol, thiomersal, or mixtures thereof. In general, an amount of from 0.001% to 2.5% (w/v), preferably from 0.002 to 1.5% (w/v) of preservative or mixture of different preservatives, based on the entire formulation, is sufficient.

In one aspect of the invention, the nanosuspension formulations must not contain a vaccine, as it has turned out that the presence of a vaccine often destroys the nanosuspension structure. In another aspect of the invention, the nanosuspension formulations are devoid of any oily component as it may destabilize the active ingredient.

Preferred embodiments of the nanosuspensions of the present invention are:

| | | |
|---|---|---|
| (i) | 2-10% (w/v) | lufenuron or hexaflumuron |
| | 1.25% | polyethoxylated castor oil (Cremophor EL) |
| | 0.05% | sodium dodecyl sulphate |
| | 0.9% | sodium chloride |
| | ad 100% | water |
| (ii) | 2-10% (w/v) | lufenuron or hexaflumuron |
| | 2.5% | PVA 7500 to 40000 |
| | 0.02% | sodium dioctylsulfosuccinate |
| | 0.9% | sodium chloride |
| | ad 100% | water |
| (iii) | 2-10% (w/v) | lufenuron or hexaflumuron |
| | 2.5% | poloxamer 188 (Pluronic ® F68) |
| | 0.02% | sodium dioctylsulfosuccinate |
| | 0.9% | sodium chloride |
| | ad 100% | water |

The nanosuspension formulations of the present invention have a small active ingredient particle size, as mentioned above, and a relatively low viscosity. Typically, the viscosity—taken at about 20° C.—is from about 500 mPas to 1 mPas, preferably from 250 mPas to 1 mPas, and more preferably from 100 mPas to 1 mPas.

Due to the specific ingredients and the physical properties as outlined above, the nanosuspension formulations of the present invention share a number of unexpected advantages, especially:
high chemical stability of the active ingredient lufenuron or hexaflumuron;
high stability of the formulation, in particular low tendency of the active ingredient to aggregate and/or form sediments;
high shelf life; the formulations may be stored for at least 1 year, preferably for at least two years.
increased bioavailability relative to a regular suspension of the active; accordingly, the dose per fish as well as the volume to be injected may be reduced and the environment is preserved.

The nanosuspension formulations may be prepared by methods known per se, for example by wet milling. To this end, the active ingredient lufenuron or hexaflumuron is dispersed in an aqueous solution comprising the surface stabilizer(s) and optionally further excipients, and the resulting suspension is milled in a suitable mill until the desired particle size of ≦1000 nm is reached. According to one embodiment, the milling is performed with a concentrated dispersion of the active ingredient, the surface stabilizer(s) and optionally further excipients in water, and the formulation is diluted after milling. In addition, the nanosuspension formulations may be sterilized afterwards, for example, by heat-sterilization.

The nanosuspension formulations of the present invention are administered to the fish by injection in a manner known per se. In case of salmonids one treatment schedule comprises treating the fish during the initial fresh water phase before transfer to sea. According to a further embodiment of the invention, the treatment is performed whilst the fish are already at sea.

The overall amount of lufenuron or hexaflumuron being injected is preferably from 10 to 200 mg per kg of fish biomass, more preferably from 15 to 100 mg per kg of fish biomass, even more preferably from 20 to 100 mg/kg fish biomass, and especially from 25 to 75 mg/kg fish biomass.

In accordance with this invention lufenuron and hexaflumuron are both excellently suited for use in the control of fish-parasitic crustaceans. These include the Family Caligidae with representative genus Dissonus, Caligus (i.e. C. curtus, C. elongatus, C. clemensi, C. rogercresseyii), and Lepeophtheirus (i.e. L. salmonis); Families Cecropidae, Dichelesthiidae, Lernaeopodidae with representative genus Salmincola; Families Pandaridae, Pennellidae with representative genus Lernaeocera and Pennella; and Family Sphyriidae; Family Lernaeidae with representative genus Lernaea; Families Bomolochidae, Chondracanthidae, Ergasilidae and Philichthyidae; Family Argulidae with representative genus Argulus (i.e. A. foliaceus).

The fish include food fish, breeding fish, aquarium, pond, river, reservoir fish of all ages occurring in freshwater, sea water and brackish water. For example, bass, bream, carp, catfish, char, chub, cichlid, cod, eel, flounder, gourami, grayling, grouper, halibut, mullet, plaice, pompano, roach, rudd, salmon, sole, tilapia, trout, tuna, whitefish, yellowtail.

Lufenuron and hexaflumuron are particularly suitable for treating salmon. The term “salmon” within the scope of this invention will be understood as comprising all representatives of the family Salmonidae, especially of the subfamily salmoninae, and preferably, the Atlantic salmon (Salmo salar), rainbow trout (Oncorhynchus mykiss), brown or sea trout (S. trutta), the Pacific salmon, Cherry salmon or seema (O. masou), Taiwanese salmon (O. masou formosanum), chinook salmon or King salmon (O. tshawytscha), chum salmon or Calico salmon (O. keta), coho salmon or silver salmon (O. kisutch), pink salmon (O. gorbuscha), Sockeye salmon or Red salmon (O. nerka), artificially propagated species, such as Salmo clarkii, and Salvelinus species such as Brook trout (S. fontinalis).

Preferred objects of the present invention are the Atlantic and Pacific salmon and the sea trout including trout species, which are farmed at sea but not traditionally called “sea trout”.

When applying lufenuron or hexaflumuron according to the present invention, the fish will absorb the active ingredient such that a therapeutically effective concentration of the active substance will be maintained for a prolonged time, for example for at least 5 months, preferably for at least 6 months and more preferably for at least 9 months time. Trials have shown that the protection period of the fish against sea lice corresponds very well with the observed lufenuron levels in the fish fillet or blood.

While the treatment with lufenuron or hexaflumuron alone according to the treatment of the present invention provides in general a complete protection against sea lice for extended periods of time, said treatment may in certain circumstances be further improved by an additional and separate treatment with either another sea lice controlling agent; or a vaccine component including immune enhancing agents; or a feed ingredient containing immune modifying agents. Such combination treatments might be required where the fish have already been infested with parasites, which have matured before the lufenuron or hexaflumuron treatment, or in case rapid clearance of the parasites is desired.

Suitable further sea lice controlling agents are, for example, hydrogen peroxide; form-aldehyde; an organophosphate such as trichlorfon, malathion, dichlorvos or azamethiphos; a macrocyclic lactone such as ivermectin, emamectin benzoate or moxidectin; a pyrethroid such as cypermethrin, in particular cypermethrin cis-40: trans-60 or high cis cypermethrin cis-80: trans-20, or deltamethrin; a neonicotinoid such as imidacloprid, nitenpyram, thiametoxam or thiacloprid; a spinosyn such as spinosad; an insect juvenile hormone analogue such as epofenonane, triprene, methoprene, hydroprene or kinoprene; or a carbamate such as phenoxycarb.

If the treatment with a nanosuspension formulation according to the present invention is complemented with an additional treatment with another compound being active in the control of sea lice, said combination partner is preferably an organophosphate, a pyrethroid such as cypermethrin or deltamethrin, a macrocyclic lactone such as emamectin benzoate; hydrogen peroxide; or a neonicotinoid such as thiacloprid.

A suitable combination treatment with a nanosuspension formulation of the present invention and another sea lice-controlling agent may be performed, for example, by treating the fish, in particular salmon, initially with lufenuron or hexaflumuron by injection as described above, and thereafter, for example 3 months, preferably 5 months, more preferably 6 months and in particular 8 months following the end of the lufenuron or hexaflumuron treatment performing a treatment with the additional sea lice controlling agent; said second treatment may be a bath treatment, an in-feed treatment or preferably a treatment by injecting the additional sea lice controlling agent to the fish. According to a preferred embodiment of this combination treatment, the treatment per injection with lufenuron or hexaflumuron takes place at the end of the fresh water phase of salmon evolution or at the beginning of their sea water phase.

A further combination treatment comprises first of all treating the fish, in particular salmon, with the additional sea lice controlling agent and thereafter, for example 1 hour to 2 months thereafter, preferably 1 hour to 1 month thereafter or in particular 1 week to 1 month thereafter, performing a lufenuron or hexaflumuron injection treatment according to the present invention as described above. According to a preferred embodiment of this combination treatment, the treatment with the additional sea lice controlling agent is a bath treatment, an in-feed treatment or injectable treatment which takes place at the beginning of the sea water phase, for example 1 hour to 3 months, preferably 6 hours to 2 months and in particular 12 hours to 1 month following the release of the fish to sea water.

According to a further embodiment of the invention, the treatment via injection with lufenuron or hexaflumuron is combined with a separate vaccination of the fish against typical bacterial or viral infections. Typical bacterial diseases to be treated by vaccination are, for example, vibrosis, furunculosis, wound diseases, atypical aeromonas salmonicida, piscirickettsiosis or ERM/yersiniosis. Examples of viral diseases to be treated are pancreas disease/PDV, infectious pancreatic necrosis/IPNV or infectious salmon anemia/ISAV. The vaccine is in general applied by a bath or in-feed treatment or preferably by injection. The vaccination may take place either shortly before, during or after the lufenuron or hexaflumuron injection treatment of the fish, but is always separated from the lufenuron or hexaflumuron injection.

According to still a further embodiment of the invention, the injection treatment with lufenuron or hexaflumuron is combined with a separate treatment with a feed ingredient which has modulatory effect on the biology, physiology, biochemistry and, particularly, immunology of the fish. Typical functions might be increased secretion of mucus, or changes in the characteristics of the mucus, such that the exposure of the parasites to the lufenuron or hexaflumuron is enhanced, or the lice exposed to the lufenuron or hexaflumuron are less able to adhere to the treated fish. The use of the modulatory in-feed ingredient may take place over extended periods in the fish production cycle or may take place before or after the treatment with the nanosuspension formulation of the present invention. Examples of such modulatory ingredients are glucans, mannans or alginates either used alone or in combination with vitamins and/or minerals.

The following Examples further illustrate the invention.

### Example 1

### (a) Preparation of a Nanosuspension

A suspension was prepared comprising 10%(w/v) lufenuron, 1.25% Cremophor EL (reaction product of castor oil and ethylene oxide in a molar ratio of about 1:35), 0.04% (w/v) sodium dodecyl sulphate, and 0.9% saline ad 100% (w/v), and was wet-milled, until an average particle size of ≦600 nm was reached. 0.9% saline was then added until the concentration of active ingredient and excipients was divided in halves.

### (b) In Vivo Test in Atlantic Salmon

Three groups of 150 salmon (Atlantic Salmon, S. salar) of an average weight 200 g were injected with an amount of the above nanosuspension (a) equivalent to 25 mg (cage 1), 50 mg (cage 2) or 75 mg lufenuron per kg salmon (cage 3). A fourth cage containing 150 salmon of average weight 200 g was added to the study as a non-treated control group.

After treatment, the fish were exposed to natural sea lice infestation for a period of 8 months. During the study the water temperature varied between 4 and 12° C. Sea louse numbers were assessed on a monthly basis until month 8. Counts were conducted on 10 fish at each sampling occasion. The numbers of pre-adults and adult L. salmonis were recorded for each fish. Efficacy was calculated using the formula:

% Efficacy=100−(100×mean of treatment group/mean of control).

TABLE 1
Efficacy Results pre-adult and adult count

| | % Efficacy | % efficacy | % efficacy |
|---|---|---|---|
| | 25 mg/kg | 50 mg/kg | 75 mg/kg |
| Time | lufenuron | lufenuron | lufenuron |
| 1 month | 100 | 90 | 100 |
| 2 month | 95 | 100 | 100 |
| 3 month | 100 | 100 | 100 |
| 4 month | 96 | 100 | 96 |
| 5 month | 100 | 97 | 100 |
| 6 month | 81 | 96 | 100 |
| 7 month | — | 98 | 98 |
| 8 month | — | 98 | 100 |

As shown in Table 1, the 25 mg/kg treatment provided complete protection of the fish against sea lice infestation for at least 5 month, whereas the complete protection with 50 mg/kg fish and 75 mg/kg even lasted until the end of the study (8 month).

Similar results are obtained if the study is repeated with corresponding nanosuspension formulations containing an equivalent amount of hexaflumuron instead of lufenuron.

1. Stable aqueous nanosuspension dosage formulation for injection to fish, comprising
(i) an active ingredient which is lufenuron or hexaflumuron, each in form of particles having an effective average particle size of ≦1000 nm;
(ii) one or more surface stabilizers selected from the group consisting of a surfactant and a polymer or a mixture thereof; and
(iii) water.

2. The dosage formulation according to claim 1, wherein the active ingredient is lufenuron.

3. The dosage formulation according to claim 2, wherein the concentration of active ingredient is from 3 to 8% (w/v), based on the entire formulation.

4. The dosage formulation according to claim 3, wherein the surface stabilizer comprises one or more anionic, non-ionic or zwitterionic surfactants selected from the group consisting of an organic sulfonic acid comprising from 8 to 24 C-atoms or a salt thereof; a fatty acid comprising from 4 to 28 C-atoms or a salt thereof; a fatty alcohol; a sorbitan fatty acid ester; a polyoxyethylene sorbitan fatty acid ester; a polyethoxylated castor oil; a polyoxyethylene ester of a fatty acid; glycerol monostearate; a poloxamer, lecithin, a hydroxylated lecithin and a phosphatidyl choline.

5. The dosage formulation according to claim 4, wherein the surface stabilizer comprises one or more anionic or non-ionic surfactants selected from the group consisting of sodium cetylstearyl sulphate, sodium lauryl sulphate, sodium dodecyl sulphate, sodium dioctylsulfosuccinate, oleic acid, sodium oleate, sodium deoxycholate, stearic acid, calcium stearate, cetyl alcohol, stearyl alcohol, cetostearyl alcohol, sorbitan monopalmitate, sorbitan monostearate, sorbitan sesquioleate, sorbitan trioleate, polyoxyethylene(20)-sorbitan monolaurate, polyoxyethylene(20)-sorbitan monopalmitate, polyoxyethylene(20)-sorbitan monostearate, polyoxyethylene(20)-sorbitan monooleate, a reaction product of castor oil and ethylene oxide in a molar ratio of about 1:35, a polyoxyethylene stearate, a polyoxyethylene 12-hydroxy-stearate, a poloxamer having an average molecular weight Mw of from 1500 to 4000 and having an polyoxyethylene/polyoxypropylene ratio from 70/30 to 80/20.

6. The dosage formulation according to claim 5, wherein the surface stabilizer comprises one or more anionic or non-ionic surfactants selected from the group consisting of sodium dodecyl sulphate, sodium dioctylsulfosuccinate, a reaction product of castor oil and ethylene oxide in a molar ratio of about 1:35, polyoxyethylene(20)-sorbitan monolaurate, polyoxyethylene(20)-sorbitan monopalmitate, and a poloxamer having an average molecular weight Mw of from 1500 to 4000 and having an polyoxyethylene/polyoxypropylene ratio from 70/30 to 80/20.

7. The dosage formulation according to claim 3, wherein the surface stabilizer comprises one or more polymers selected from the group consisting of a polyvinylpyrrolidones, a polylactic acid, a polylactic-co-glycolic acid; a polyethylene glycol, a natural and modified cellulose, a starch, a polysaccharide, a polydimethylsiloxane, a silicon dioxide, tragacanth, albumin, hydrolyzed gelatin and cholesterol.

8. The dosage formulation according to claim 7, wherein the surface stabilizer comprises one or more polymers selected from the group consisting of a polyvinyl alcohol (PVA) having an average molecular weight Mw of from 7500 to 40000 and a polyvinyl pyrrolidone (PVP) having an average molecular weight Mw of from about 2500 to 50000.

9. The dosage formulation according to claim 7, wherein the surface stabilizer comprises one or more polymers and/or polymers selected from the group consisting of sodium dodecyl sulphate, sodium dioctylsulfosuccinate, a reaction product of castor oil and ethylene oxide in a molar ratio of about 1:35, polyoxyethylene(20)-sorbitan monolaurate, polyoxyethylene(20)-sorbitan monopalmitate, a poloxamer having an average molecular weight Mw of from 1500 to 4000 and having an polyoxyethylene/polyoxypropylene ratio from 70/30 to 80/20, a polyvinyl alcohol (PVA) having an average molecular weight Mw of from 7500 to 40000 and a polyvinyl pyrrolidone (PVP) having an average molecular weight Mw of from about 2500 to 50000.

10. The dosage formulation according to claim 9, comprising, in addition a crystal growth inhibitor, selected from the group consisting of a mono- or disaccharide, a sugar alcohol, a glycol, glycerol, a water-soluble salt, and mixtures thereof.

11. The dosage formulation according to claim 10, comprising from 1 to 20% (w/v) active ingredient, from 0.5 to 20% (w/v) of one or more surface stabilizers, from 0 to 20% (w/v) of a crystal growth inhibitor and water.

12. The dosage formulation according to claim 11, consisting essentially of from 2 to 10% (w/v) lufenuron, from 1.0 to 8% (w/v) of one or more surface stabilizers, from 0.25 to 15% (w/v) of a crystal growth inhibitor and water ad 100%.

13. The dosage formulation according to claim 11, comprising an average particle size of ≦600 nm.

14. The dosage formulation according to claim 13, which is prepared by dispersing the active ingredient in an aqueous solution comprising the one or more surface stabilizers and optionally further excipients, and milling the resulting suspension in a mill until the desired particle size is reached.

15. The dosage formulation according to claim 14 for eliminating, reducing or preventing sea lice in a fish population, which comprises injecting said dosage formulation to the fish at a dose of from 15 to 100 mg active ingredient per kg of fish biomass.

16. The dosage formulation according to claim 10 comprising sodium chloride as crystal growth inhibitor.

17. The dosage formulation according to claim 13 comprising an average particle size of from 10 to 600 nm.

18. The dosage formulation according to claim 14 for eliminating, reducing or preventing sea lice in a fish population, which comprises injecting said dosage formulation to the fish at a dose of from 25 to 75 mg active ingredient per kg of fish biomass.
